(19) United States
(12) Patent Application Publication
(10) Pub. No.: US 2025/0177744 A1
(43) Pub. Date: Jun. 5, 2025

(54) FOOT ELECTRICAL STIMULATION MASSAGER AND MASSAGE DEVICE

(71) Applicant: CHANGSHA ANXIANG MEDICAL TECHNOLOGY CO., LTD., CHANGSHA (CN)

(72) Inventor: JUNYUAN CHEN, CHANGSHA (CN)

(21) Appl. No.: 18/404,916

(22) Filed: Jan. 5, 2024

(30) Foreign Application Priority Data
Dec. 5, 2023 (CN) 202323306521.3

(51) Int. Cl. A61N 1/36; A61N 1/04

(52) U.S. Cl. CPC A61N 1/36003; A61N 1/0408; A61N 1/0472; A61N 1/36014

(57) ABSTRACT

A foot electrical stimulation massager includes: a first conductive region and a second conductive region, the first conductive region and the second conductive region being configured to simultaneously apply electrical stimulation to a same sole; wherein the first conductive region is connected to a positive electrode of a power source, the second conductive region is connected to a negative electrode of the power source, the first conductive region is configured to receive a current input by the positive electrode of the power source and transmit the current to the second conductive region via the sole upon transmitting the current to the sole thereon to apply the electrical stimulation, and the second conductive region is configured to output the current to the negative electrode of the power source.

### CROSS-REFERENCE TO RELATED APPLICATIONS

This application claims priority to Chinese Patent Application No. 202323306521.3, filed with the Chinese Patent Office on Dec. 5, 2023, titled “FOOT ELECTRICAL STIMULATION MASSAGER AND MASSAGE DEVICE”, the entire contents of which are incorporated herein by reference.

### TECHNICAL FIELD

Embodiments of the present disclosure relate to the technical field of massagers, and in particular, relate to a foot electrical stimulation massager and a massage device.

### BACKGROUND

In recent years, people often suffer from anxieties and physical and mental fatigue due to the fast-paced lifestyle and various aspects of life pressure. Electrical stimulation massage for feet is a common way to relieve fatigue. There are many acupoints on the sole of a human body. The massage for the foot to stimulate the acupoints on the foot can promote the blood circulation of the body, accelerate metabolism, help the body relax, relieve anxieties, and mitigate physical fatigue. A foot electrical stimulation massager is easy to operate, easy to use, and highly popular among people.

However, a conventional foot electrical stimulation massager needs to perform electrical stimulation massage on both feet at the same time, wherein one foot is in contact with the positive electrode of the massage region and the other foot is in contact with the negative electrode of the massage region, and the current transmitted by the foot electrical stimulation massager needs to pass from one foot to the other foot through the human torso, which also results in some potential safety hazards and a poor physical therapy effect.

### SUMMARY

Embodiments of the present disclosure provide a foot electrical stimulation massager. The foot electrical stimulation massager includes: a first conductive region and a second conductive region, the first conductive region and the second conductive region being configured to simultaneously apply electrical stimulation to a same sole; wherein the first conductive region is connected to a positive electrode of a power source, the second conductive region is connected to a negative electrode of the power source, the first conductive region is configured to receive a current input by the positive electrode of the power source and transmit the current to the second conductive region via the sole upon transmitting the current to the sole thereon to apply the electrical stimulation, and the second conductive region is configured to output the current to the negative electrode of the power source.

In an optional embodiment, the foot electrical stimulation massager further includes: a positive electrode connection terminal and a negative electrode connection terminal; wherein the positive electrode connection terminal is electrically connected to the first conductive region, and the positive electrode connection terminal is configured to be connected to the positive electrode of the power source; and the negative electrode connection terminal is electrically connected to the second conductive region, and the positive electrode connection terminal is configured to be connected to the negative electrode of the power source.

In an optional embodiment, the foot electrical stimulation massager further includes: a base; wherein the first conductive region, the second conductive region, the positive electrode connection terminal, and the negative electrode connection terminal are all disposed on a surface of the base.

In an optional embodiment, the first conductive region is in contact with a front plantar region of the sole in use, and the second conductive region is in contact with a heel region of the sole in use.

In an optional embodiment, the foot electrical stimulation massager further includes: a third conductive region and a fourth conductive region, the third conductive region and the fourth conductive region being configured to simultaneously apply electrical stimulation to another sole; wherein the third conductive region is electrically connected to the positive electrode connection terminal, and the fourth conductive region is electrically connected to the negative electrode connection terminal.

In an optional embodiment, the positive electrode connection terminal and the negative electrode connection terminal are disposed between the first conductive region and the third conductive region.

In an optional embodiment, the foot electrical stimulation massager further includes: a foot massage cushion; wherein the first conductive region and the second conductive region are disposed on an outer surface of the foot massage cushion.

In an optional embodiment, the foot electrical stimulation massager is a slipper, the slipper including an insulative shoe sole; wherein the first conductive region and the second conductive region are both disposed on a surface, close to the sole, of the insulative shoe sole.

In an optional embodiment, the positive electrode connection terminal is disposed at an end corner, facing towards the second conductive region, on the first conductive region; and the negative electrode connection terminal is disposed at an end corner, opposite to the positive electrode connection terminal, on the second conductive region.

Embodiments of the present disclosure further provide a massage device. The massage device includes the foot electrical stimulation massager according to any of the above embodiments.

### BRIEF DESCRIPTION OF THE DRAWINGS

By reading the detailed description of preferred embodiments hereinafter, various other advantages and beneficial effects become clear and apparent for persons of ordinary skill in the art. The accompanying drawings are merely for illustrating the preferred embodiments, but shall not be construed as limiting the present disclosure. In all the accompanying drawings, like reference numerals denote like parts. In the drawings:

FIG. 1 is a perspective view of a foot electrical stimulation massager according to an embodiment of the present disclosure;

FIG. 2 is a schematic structural view of a sole involved according to an embodiment of the present disclosure;

FIG. 3 is a schematic structural view of a foot electrical stimulation massager according to an embodiment of the present disclosure;

FIG. 4 is a schematic structural view of a slipper according to an embodiment of the present disclosure; and

FIG. 5 is a schematic structural view of a slipper according to an embodiment of the present disclosure.

Reference numerals in the embodiments and denotations thereof:
100—foot electrical stimulation massager; 200—sole; 400—slipper;
11—first conductive region; 12—second conductive region; 13—third conductive region; 14—fourth conductive region; 15—positive electrode connection terminal; 16—negative electrode connection terminal; 17—base; 18—conductive region;
21—front plantar region; 22—heel region; 23—lateral region; 24—plantar arch region;
30—foot massage cushion;
41—insulative shoe sole; 42—protrusion; 43—upper vamp.

### DETAILED DESCRIPTION

The embodiments containing the technical solutions of the present disclosure are described in detail with reference to the accompanying drawings. The embodiments hereinafter are only used to clearly describe the technical solutions of the present disclosure. Therefore, these embodiments are only used as examples, but are not intended to limit the protection scope of the present disclosure.

Unless defined otherwise, all technical and scientific terms used herein have the same meaning as commonly understood by one of ordinary skill in the art to which the present disclosure pertains. The terms used herein in the specification of present disclosure are only intended to illustrate the specific embodiments of the present disclosure, instead of limiting the present disclosure. The terms “comprise,” “include,” and any variations thereof in the specification, claims, and the description of the drawings of the present disclosure are intended to cover a non-exclusive inclusion.

In the description of the present disclosure, the terms “first,” “second,” and the like are only used for distinguishing different objects, but shall not be understood as indication or implication of relative importance or implicit indication of the number of the specific technical features, the specific sequences, or priorities. In the description of the embodiments of the present disclosure, the term “multiple” or “a plurality of” signifies at least two, unless otherwise specified.

The terms “example” and “embodiment” in this specification signify that the specific characteristics, structures, or features described with reference to the embodiments may be covered in at least one embodiment of the present disclosure. This term, when appearing in various positions of the description, neither indicates the same embodiment, nor indicates an independent or optional embodiment that is exclusive of the other embodiments. A person skilled in the art would implicitly or explicitly understand that the embodiments described in this specification may be incorporated with other embodiments.

In the description of the embodiments of the present disclosure, the term “and/or” is merely an association relationship for describing associated objects, which represents that there may exist three types of relationships, for example, A and/or B may represent three situations: only A exists, both A and B exist, and only B exists. In addition, the forward-slash symbol “/” generally represents an “or” relationship between associated objects before and after the symbol.

In the description of the embodiments of the present disclosure, the term “multiple” or “a plurality of” signifies more than two (including two), unless otherwise specified. Likewise, the term “a plurality of groups” or “multiple groups” signifies more than two groups (including two groups), and the term “a plurality of pieces” or “multiple pieces” signifies more than two pieces (including two pieces).

In the description of the embodiments of the present disclosure, it should be understood that the terms “central,” “transversal,” “longitudinal,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” “circumferential,” and the like indicate orientations and position relationships which are based on the illustrations in the accompanying drawings, and these terms are merely for ease and brevity of the description, instead of indicating or implying that the devices or elements shall have a particular orientation and shall be structured and operated based on the particular orientation. Accordingly, these terms shall not be construed as limiting the present disclosure.

In the description of the embodiments of the present disclosure, it should be noted that unless otherwise specified and defined, the terms “mounted,” “coupled,” “connected,” “secured,” and derivative forms thereof shall be understood in a broad sense, which, for example, may be understood as secured connection, detachable connection or integral connection; may be understood as mechanical connection or electrical connection, or understood as direct connection, indirect connection via an intermediate medium, or communication between the interiors of two elements or interactions between two elements. Persons of ordinary skill in the art may understand the specific meanings of the above terms in the embodiments of the present disclosure according to the actual circumstances and contexts.

Projection regions of five ZANG-organs and six FU-organs and other various tissues and organs are distributed in a human sole, such as heart, lung, kidney, liver, gallbladder, stomach, and the like. Stimulating the corresponding projection regions regulates functions of the corresponding tissues and organs, and ZANG and FU-organs. Foot massage stimulates the corresponding projection regions of the sole, promotes blood circulation, dredges imbalance of the human metabolism function, helps to excrete toxins in the body, and further promotes the immune ability and metabolism of the human body, helps to improve blood pressure, keeps the intestinal tract smooth, and improves sleep quality.

The foot electrical stimulation massager transmits an electric current to the sole, stimulates the projection regions in the soles, and achieves the massage effect. The inventors of the present disclosure have found that the conventional foot electrical stimulation massager is mainly based on a foot massage cushion. An outer surface of the foot massage cushion is provided with two sole-shaped conductive regions, a positive electrode connection terminal, and a negative electrode connection terminal. One of the conductive regions is electrically connected to the positive electrode connection terminal, and the other conductive region is electrically connected to the negative electrode connection terminal. During use, the soles of two feet are placed on the two conductive regions respectively, such that one of the soles is in contact with one of the conductive regions, the other of the soles is in contact with the other of the conductive regions, the positive electrode connection terminal is electrically connected to the positive electrode of a power source, and the negative electrode connection terminal is electrically connected to the negative electrode of the power source, such that current electrically stimulates the soles in contact with the conductive regions.

However, the current is input to the conductive region via the positive electrode connection terminal of the power source, the current is transmitted to the human torso via the sole after the electrical stimulation is performed by the sole transmitted to the conductive region, the current is transmitted to the other conductive region via the sole after the electrical stimulation is performed by the human torso, and then the current is output to the negative electrode of the power source via the negative electrode connection terminal. After the current passes through the human body, electrolysis, electrophoresis and electroosmosis of liquid substances in the body may occur, and a variety of electrolysis products such as acid and alkali may be produced, which may affect protein metabolism and cell permeability. As a result, damages may be caused to the heart, lung, nervous system and other organs of the human body, arrhythmia, respiratory depression, and other manifestations may also be caused, and even death may be caused in some extremely severe cases. Although the electric current transmitted by the foot electrical stimulation massager is very small, when the foot electrical stimulation massager is used, the electric current needs to pass through the human torso to form a current loop to realize the electrical stimulation of the sole, which has a potential safety hazard and may cause damage to the human body. In addition, two soles need to be placed in different conductive regions at the same time, and hence the electrical stimulation of only one sole cannot be realized. In addition, the sole of the human body has a larger area and a smaller width at the ankle, and when an electric current passes through the ankle, the electric current converges, such that the best electrical stimulation effect is on the ankle, rather than on the sole, and thus the electrical stimulation effect is poor.

In order to solve the problems that the foot electrical stimulation massager has a potential safety hazard and has a poor effect on the foot, the inventors of the present disclosure, after intensive research, has designed a foot electrical stimulation massager, wherein a conductive region for performing electrical stimulation on the same sole is separated to form two sub-conductive regions, and during use, one sole is placed on the two sub-conductive regions, wherein one of the sub-conductive regions is used for receiving the current input by the positive electrode of a power source, the current is transmitted to the sole for electrical stimulation, then the current is transmitted to the other of the sub-conductive regions through the sole, and the current is output from the sub-conductive region to the negative electrode of the power source. With this design, a current loop may be formed when the electric current passes through the sole without passing through the human torso, which is safer, and the electric current only stimulates the sole without passing through the ankle. Therefore, a better electrical stimulation is achieved on the sole. In addition, the power source, the two sub-conductive regions and one sole form a current loop. In this case, electrical stimulation may be carried out on one single sole separately. Accordingly, this foot electrical stimulation massager is more adaptable and more versatile in use.

The structure of the foot electrical stimulation massager disclosed in the embodiments of the present disclosure can be applied not only to a foot massage apparatus, but also to any electronic or physiotherapy apparatus having a similar function, such as a plantar physiotherapy apparatus and a foot pulse therapeutic apparatus. The embodiments of the present disclosure are described only by taking the foot electrical stimulation massager as an example.

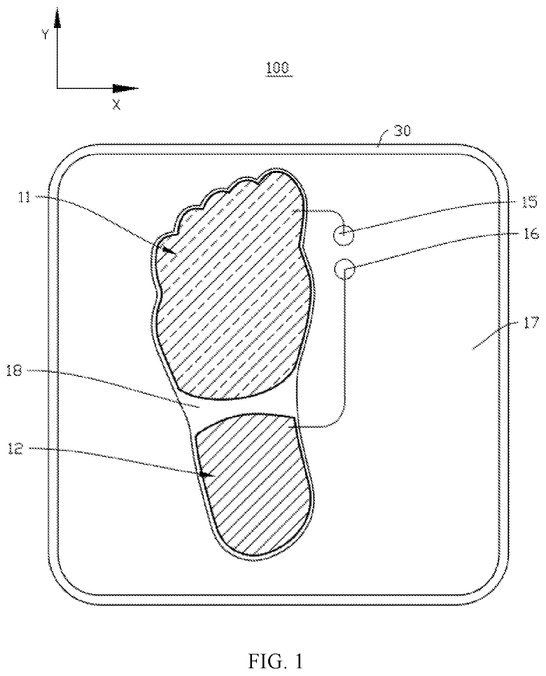

Referring to FIG. 1, FIG. 1 is a perspective view of a foot electrical stimulation massager 100 according to an embodiment of the present disclosure. The foot electrical stimulation massager 100 includes: a first conductive region 11 and a second conductive region 12. The first conductive region 11 and the second conductive region 12 are configured to simultaneously apply electrical stimulation to a same sole. The first conductive region 11 is connected to a positive electrode of a power source. The second conductive region 12 is connected to a negative electrode of the power source. The first conductive region 11 is configured to receive a current input by the positive electrode of the power source and transmit the current to the second conductive region via the sole upon transmitting the current to the sole thereon to apply the electrical stimulation. The second conductive region 12 is configured to output the current to the negative electrode of the power source.

The foot electrical stimulation massager 100 stimulates the corresponding projection areas on the sole by generating a pulse current at a specific frequency, thereby achieving the effects of promoting blood circulation and relieving fatigue. Electrical stimulation promotes stimulation of a nervous system, enhance contraction and relaxation of muscles, improves blood circulation and metabolism, thereby achieving achieve the effect of relaxing muscle and relieving pain. The foot electrical stimulation massager 100 simulates nerve signals in a natural physiological process of a human body by generating electric pulse signals with a specific frequency, amplitude and width, stimulates nerve endings on acupoints on the sole of the human body, and transmits the same to the brain through the nervous system, and causes corresponding physiological responses. The foot electrical stimulation massager 100 may be in various forms, which may be, for example, an electrical muscle stimulation (EMS) foot massage cushion or an electrical stimulation massage slipper.

In the foot electrical stimulation massager 100, the first conductive region 11 and the second conductive region 12 are main components supplying electrical stimulation to the foot. The first conductive region 11 and the second conductive region 12 may be respectively formed by arranging printed circuits on a surface of an insulative base. For example, a silver paste may be printed on a surface of an insulative rubber such that conductive regions are formed on the surface of the insulative rubber. Conductive regions may also be formed directly from conductive materials, such as conductive rubber, graphene, and the like. The first conductive region 11 and the second conductive region 12 are two independent regions and are not directly conducted to each other. The first conductive region 11 and the second conductive region 12 may be two independent regions on one component or may be two components separately. The first conductive region 11 and the second conductive region 12 may be vertically arranged, i.e., arranged in a Y-axis direction, or may be horizontally arranged, i.e., arranged in an X-axis direction.

As illustrated in FIG. 1, the first conductive region 11 and the second conductive region 12 combine to form a conductive region 18 that simultaneously electrically stimulates the same sole. The first conductive region 11 and the second conductive region 12 may have the same shape or may have different shapes. For example, the first conductive region 11 and the second conductive region 12 may both have a semi-elliptical shape or may have two different irregular patterns. The first conductive region 11 and the second conductive region 12 may together form a shape capable of accommodating a sole, for example, a sole shape. It should be noted that the foot electrical stimulation massager 100 may be provided with only one conductive region 18 for electrical stimulation of only one sole; or a plurality of conductive regions 18 may be provided for electrical stimulation of multiple soles. When the foot electrical stimulation massager 100 is employed to apply electrical stimulation to multiple pairs of soles, a plurality of conductive regions 18 may be arranged side by side along the X-axis direction or along the Y-axis direction on the foot electrical stimulation massager 100 to form a conductive region array.

During use of the foot electrical stimulation massager 100, after the first conductive region 11 is electrically connected to the positive electrode of the power source and the second conductive region 12 is electrically connected to the negative electrode of the power source, one sole is placed on the conductive region 18 formed by the combination of the first conductive region 11 and the second conductive region 12, such that part of the sole is in contact with the first conductive region 11 and part of the sole is in contact with the second conductive region 12. The power switch is turned on, and the positive electrode of the power source transmits a current to the first conductive region 11; after the first conductive region 11 transmits the current to the sole in contact therewith for electrical stimulation, the current is transmitted to the second conductive region 12 via the sole, and then the current is transmitted to the negative electrode of the power source via the second conductive region 12.

According to the above embodiments, the first conductive region and the second conductive region are made to electrically stimulate the same sole. With this design, a current loop may be formed when the electric current passes through the sole without passing through the human torso, which is safer, and the electric current only stimulates the sole without passing through the ankle. Therefore, a better electrical stimulation is achieved on the sole. In addition, the power source, the first conductive region, the second conductive region, and the sole form a current loop. In this case, electrical stimulation may be carried out on one single sole separately. Accordingly, this foot electrical stimulation massager is more adaptable and more versatile in use.

In some embodiments of the present disclosure, as illustrated in FIG. 1, the foot electrical stimulation massager 100 further includes a positive electrode connection terminal 15 and a negative electrode connection terminal 16. The positive electrode connection terminal 15 is electrically connected to the first conductive region 11, and the positive electrode connection terminal 15 is configured to be connected to the positive terminal of the power source. The negative electrode connection terminal 16 is electrically connected to the second conductive region 12, and the negative electrode connection terminal 16 is configured to be connected to the negative electrode of the power source.

The positive electrode connection terminal 15 is configured to be connected between the positive terminal of the power source and the first conductive region 11. The positive electrode connection terminal 15 may be arranged on the first conductive region 11, and directly electrically connected to the first conductive region 11; or may be arranged separately from the first conductive region 11, and electrically connected to the first conductive region 11 via printed circuit traces, wire connections, or the like. When the positive electrode connection terminal 15 is arranged separately from the first conductive region 11, the positive electrode connection terminal 15 may be arranged in the vicinity of the first conductive region 11 or at one of end corners of the foot electrical stimulation massager 100, which depends on actual needs. The positive electrode connection terminal 15 may be of various types, for example, a conductive snap-fit structure, a pin connection structure, or the like. The positive electrode connection terminal 15 may be directly electrically connected to a connection structure corresponding to the positive electrode of the power source, or may be connected via a wire. That is, one end of the wire is electrically connected to the positive electrode connection terminal 15, and the other end is electrically connected to the positive electrode of the power source. The positive electrode connection terminal 15 may be in various shapes, for example, a circular shape, a rectangular shape, an oval shape, or the like.

The negative electrode connection terminal 16 is configured to connect the negative terminal of the power source to the second conductive region 12. The negative electrode connection terminal 16 may be arranged on the second conductive region 12, and directly electrically connected to the second conductive region 12; or may be arranged separately from the second conductive region 12, and electrically connected to the second conductive region 12 via printed circuit traces, wire connections, or the like. When the negative electrode connection terminal 16 is arranged separately from the second conductive region 12, the negative electrode connection terminal 16 may be arranged in the vicinity of the first conductive region 12 or at one of the end corners of the foot electrical stimulation massager 100, or may be arranged opposite to the positive electrode connection terminal 15, which depends on actual needs. The negative electrode connection terminal 16 may be of various types, for example, a conductive snap-fit structure, a pin connection structure, or the like. The negative electrode connection terminal 16 may be directly electrically connected to a connection structure corresponding to the negative electrode of the power source, or may be connected via a wire. That is, one end of the wire is electrically connected to the negative electrode connection terminal 16, and the other end is electrically connected to the negative electrode of the power source. The negative electrode connection terminal 16 may be in various shapes, for example, a circular shape, a rectangular shape, an oval shape, or the like.

In addition, the positive electrode connection terminal 15 and the negative electrode connection terminal 16 may also be independent connectors, and are respectively electrically connected to the first conductive region 11 and the second conductive region 12 via wires. During use, the positive electrode connection terminal 15 and the negative electrode connection terminal 16 are respectively plugged with corresponding connectors on the power source.

According to the above embodiments, the positive electrode connection terminal and the negative electrode connection terminal are arranged, such that the connection between the first conductive region and the positive electrode of the power source, and the connection between the second conductive region and the negative electrode of the power source are more convenient and simpler. In this way, the conductive regions are quickly connected to the power source.

In some embodiments of the present disclosure, as illustrated in FIG. 1, the foot electrical stimulation massager 100 further includes a base 17. The first conductive region 11, the second conductive region 12, the positive electrode connection terminal 15, and the negative electrode connection terminal 16 are all arranged on the surface of the base 17. The base 17 is a carrier for the foot electrical stimulation massager 100 to accommodate functional components. To ensure users' safety, the base 17 is typically made of an insulative material. The base 17 may be in various shapes, for example, a square shape, a rectangular shape, a circular shape, or the like, which may be designed according to actual needs. During use of the foot electrical stimulation massager 100, the surface of the base 17 where the first conductive region 11, the second conductive region 12, the positive electrode connection terminal 15, and the negative electrode connection terminal 16 are arranged is made to face upwards. In this case, various functional components arranged on the surface of the substrate 17 are visible, which is more convenient for use of the massager.

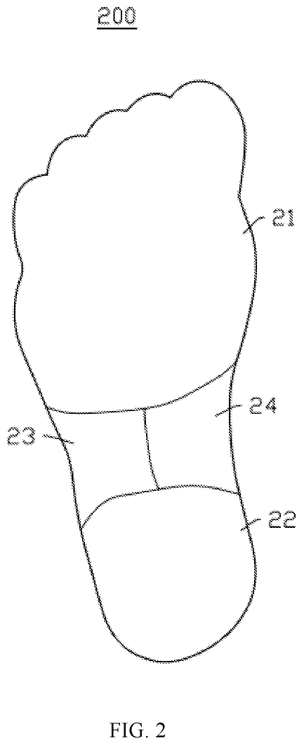

In some embodiments of the present disclosure, as illustrated in FIGS. 1 and 2, FIG. 2 is a schematic structural view of a sole involved according to an embodiment of the present disclosure. During use, the first conductive region 11 is in contact with a front plantar region 21 of the sole 200, and the second conductive region 12 is in contact with a heel region 22 of the sole 200.

The sole 200 is a part of the human foot that is in contact with the ground, i.e., the bottom of the foot. As illustrated in FIG. 2, the sole 200 is largely divided into four regions, the front plantar region 21, the heel region 22, a lateral region 23, and a plantar arch region 24. The plantar arch region 24 is a position of the arch of the foot, and often has a specific degree of curvature. The degree of curvature of the plantar arch region 24 varies from person to person. Therefore, generally, only the anterior plantar region 21, the heel region 22 and the lateral region 23 of the sole 200 are in contact with the ground. However, in order to adapt to different human bodies, the foot electrical stimulation massager 100 tends to arrange the first conductive region 11 and the second conductive region 12 on the same plane, such that the sole 200 is mainly in contact with the first conductive region 11 or the second conductive region 12 by the front plantar region 21, the heel region 22, and the lateral region 23.

As illustrated in FIG. 1, in the case that the first conductive region 11 and the second conductive region 12 are vertically arranged, i.e., arranged along the X-axis direction, the plantar arch area 24 fails to be in contact with the corresponding conductive region. As a result, a waste of resources at this position is caused, and less current flows through the plantar arch area 24, resulting in a poor electrical stimulation effect for the plantar arch area 24. The first conductive region 11 and the second conductive region 12 are vertically arranged, i.e., along the Y-axis direction. During use of the foot electrical stimulation massager 100, the front plantar region 21 is in contact with the first conductive region 11, and the heel region 22 is in contact with the second conductive region 12. The current is transmitted from the first conductive region 11 to the front plantar region 21 of the sole 200, then flows to the heel region 22 through the lateral region 23 and the plantar arch region 24 of the sole 200, and finally is transmitted from the heel region 22 to the second conductive region 12. Such a structure not only enables most of the areas of the first conductive region 11 and the second conductive region 12 to be in contact with the sole 200, but also enables the regions of the sole 200 to be electrically stimulated more uniformly, thereby achieving a better massage effect.

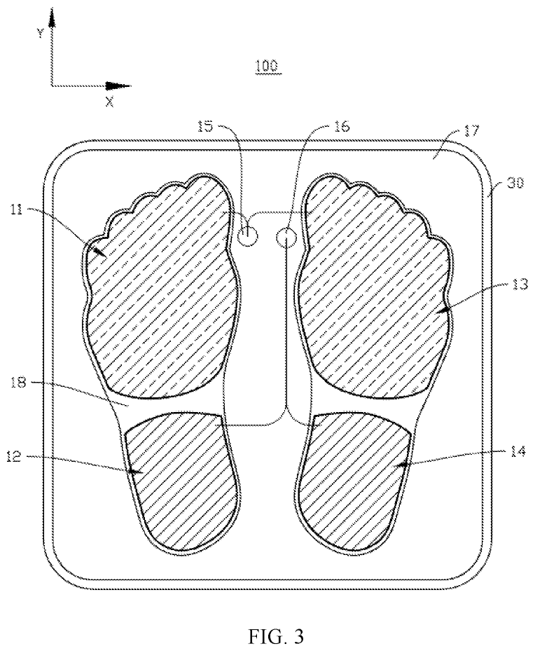

In some embodiments of the present disclosure, as illustrated in FIG. 3 which is a schematic structural diagram of another foot electrical stimulation massager 100 according to an embodiment of the present disclosure, the foot electrical stimulation massager 100 further includes a third conductive region 13 and a fourth conductive region 14. The third conductive region 13 and the fourth conductive region 14 are configured to simultaneously apply electrical stimulation to another sole. The third conductive region 13 is electrically connected to a positive electrode connection terminal 15, and the fourth conductive region 14 is electrically connected to the negative electrode connection terminal 16.

In the foot electrical stimulation massager 100, the third conductive region 13 and the fourth conductive region 14 are the main components supplying electrical stimulation to the foot, and are generally made of a conductive material, such as a metal or a carbon fiber. The third conductive region 13 and the fourth conductive region 14 may be respectively formed by arranging printed circuits on a surface of the insulative base. For example, a silver paste may be printed on a surface of an insulative rubber such that conductive regions are formed on the surface of the insulative rubber. Conductive regions may also be formed directly from conductive materials, such as conductive rubber, graphene, and the like. The third conductive region 13 and the fourth conductive region 14 are two independent regions and are not directly conducted to each other. The third conductive region 13 and the fourth conductive region 14 may be two independent regions on one component or may be two components separately. The third conductive region 13 and the fourth conductive region 14 may be vertically arranged, i.e., arranged in the direction of the Y axis, or may be horizontally arranged, i.e., arranged in the direction of the X axis.

As shown in FIG. 3, the third conductive region 13 and the fourth conductive region 14 combine to form a region that simultaneously electrically stimulates the same sole. The third conductive region 13 and the fourth conductive region 14 may have the same shape or may have different shapes. For example, the third conductive region 13 and the fourth conductive region 14 may both have a semi-elliptical shape or may have two different irregular patterns. The third conductive region 13 and the fourth conductive region 14 may together form a shape capable of accommodating a sole, for example, a sole shape. It should be noted that the foot electrical stimulation massager 100 may be provided with only one set of first conductive region 11, second conductive region 12, third conductive region 13, and fourth conductive region 14 for electrical stimulation only on one pair of soles; or may be provided with multiple sets of first conductive regions 11, second conductive regions 12, third conductive regions 13, and fourth conductive regions 14 for electrical stimulation on multiple pairs of soles. When the foot electrical stimulation massager 100 is employed to apply electrical stimulation to multiple pairs of soles, multiple sets of first conductive regions 11, the second conductive regions 12, third conductive regions 13, and fourth conductive regions 14 may be arranged side by side along the X-axis direction or along the Y-axis direction on the foot electrical stimulation massager 100 to form a conductive region array.

In addition, the first conductive region 11 and the third conductive region 13 are both electrically connected to the positive electrode connection terminal 15, and the second conductive region 12 and the fourth conductive region 14 are both electrically connected to the negative electrode connection terminal 16. During use of the foot electrical stimulation massager 100, the positive electrode of the power source is electrically connected to the positive electrode connection terminal 15, and the negative electrode of the power source is electrically connected to the negative electrode connection terminal 16. Where the power switch is turned on, the first conductive region 11 and the third conductive region 13 is electrified, and electrical stimulation may be performed on a pair of soles at the same time. Therefore, the massager is more efficient and more convenient to use.

In some embodiments of the present disclosure, as illustrated in FIG. 3, the positive electrode connection terminal 15 and the negative electrode connection terminal 16 are arranged between the first conductive region 11 and the third conductive region 13. During use of the foot electrical stimulation massager 100, it is necessary to place the foot electrical stimulation massager 100 on the ground, place one foot on the region formed by the combination of the first conductive region 11 and the second conductive region 12, and place the other foot on the region formed by the combination of the third conductive region 13 and the fourth conductive region 14. In the case that the power source is directly electrically connected to the positive electrode connection terminal 15 and the negative electrode connection terminal 16, the power source is positioned on the surface of the foot electrical stimulation massager 100. When both feet are placed on the foot electrical stimulation massager 100, sight may be blocked. In the case that the positive electrode connection terminal 15 and the negative electrode connection terminal 16 are arranged at the end corners of the foot electrical stimulation massager 100, it is difficult for a user to operate the power source. In the case that the positive electrode connection terminal 15 and the negative electrode connection terminal 16 are arranged between the first conductive region 11 and the third conductive region 13, it is only necessary to slightly open the thighs, such that the power source is directly operated and used more conveniently.

In some embodiments of the present disclosure, as illustrated in FIGS. 1 and 3, the foot electrical stimulation massager 100 includes a foot massage cushion 30. The first conductive region 11 and the second conductive region 12 are arranged on an outer surface of the foot massage cushion 30.

The foot massage cushion 30 is a main body of the foot electrical stimulation massager 100, and various functional components of the foot electrical stimulation massager 100 are arranged on the outer surface of the foot massage cushion 30. The foot massage cushion 30 is generally made of an insulative material, which may be an insulative rubber, an insulative silica gel, a plastic, or the like. The foot massage cushion 30 may have a plate shape with high hardness, i.e., it cannot be folded, or may have a soft plate shape, i.e., it can be stored by rolling, folding, and the like. The foot massage cushion 30 may be in various shapes, for example, a square shape, a rectangular shape, a circular shape, or the like. The foot electrical stimulation massager takes the foot massage cushion as the main body, such that the overall volume of the foot electrical stimulation massager is smaller, and the massager is convenient to store and convenient to carry, and has more adaptabilities to more use scenarios.

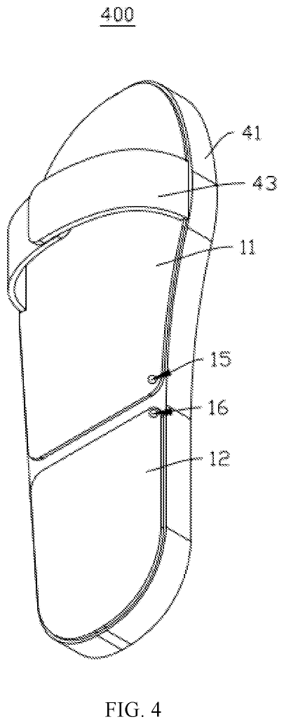

In some embodiments of the present disclosure, as illustrated in FIG. 4 which is a schematic structural view of a slipper according to an embodiment of the present disclosure, the foot electrical stimulation massager 100 is a slipper 400. The slipper 400 includes an insulative shoe sole 41, and the first conductive region 11 and the second conductive region 12 are both arranged on a side, close to the sole of the foot, of the insulative shoe sole 41.

The slipper 400 is a shoe with an empty heel, without laces or buckles, and the user may freely slide the foot into the slipper. The slipper is comfortable, light, and easy to wear. The slipper 400 mainly includes an upper vamp 43 and a shoe sole. The upper vamp 43 is configured to limit a distance between the sole and the shoe sole to ensure that the shoe sole moves along with the sole during walking. The shoe sole is positioned at a bottom of the slipper 400 for receiving a human foot. The sole includes an insulative bottom 41 made of an insulative material, and the specific material of the insulative bottom 41 may be an insulative rubber, a plastic, or the like. The first conductive region 11 and the second conductive region 12 can be separately arranged, that is, the first conductive region 11 and the second conductive region 12 are respectively and independently provided on the insulative shoe sole 41; or may be integrally arranged, that is, the first conductive region 11 and the second conductive region 12 are connected via an insulative material to form a one piece structure. The first conductive region 11 and the second conductive region 12 may be directly adhered to the surface of the insulative shoe sole 41, or a groove may be arranged in the insulative shoe sole 41, and the first conductive region 11 and the second conductive region may be arranged in the groove.

During use of the foot electrical stimulation massager 100, it is necessary to ensure that the sole is always in contact with the first conductive region 11 and the second conductive region 12. In the case that the foot electrical stimulation massager 100 is in the form of a massage cushion, during use, the position of the sole is easily shifted, such that the electrical stimulation effect is adversely affected. With regard to the slipper-shaped foot electrical stimulation massager 100, the upper vamp of the slipper restricts the position between the sole and the first conductive region 11 and the second conductive region 12, and when the sole moves, the slipper follows the sole, such that the stability of the electrical stimulation process is ensured and hence the electrical stimulation effect is better.

In some embodiments of the present disclosure, as illustrated in FIG. 4, the positive electrode connection terminal 15 is arranged at one of end corners, facing towards the second conductive region 12, of the first conductive region 11, and the negative electrode connection terminal 16 is arranged at an end corner, opposite the positive electrode connection terminal 15, of the second conductive region 12.

The position of the insulative shoe sole 41 is limited. In this case, where the positive electrode connection terminal 15 and the negative electrode connection terminal 16 are independently arranged on the insulative shoe sole 41, the area of the slipper 400 is larger, the effect achieved by the upper vamp 43 in limiting the sole and the insulative shoe sole 41 is poor, and an additional conductive channel needs to be arranged, such that the positive electrode connection terminal 15 is electrically connected to the first conductive region 11 and the negative electrode connection terminal 16 is electrically connected to the second conductive region 12. The positive electrode connection terminal 15 is arranged at an end corner of the first conductive region 11, and the negative electrode connection terminal 16 is arranged at an end corner of the second conductive region 12 corresponding to the positive electrode connection terminal 15. In this way, it is ensured that the first conductive region 11 is electrically connected to the positive electrode connection terminal 15 and the second conductive region 12 is electrically connected to the negative electrode connection terminal 16 without an additionally arranged conductive channel. In addition, the position of the insulative shoe sole 41 is not additionally occupied, such that the overall structure of the slipper is more compact. In addition, the positive electrode connection terminal 15 and the negative electrode connection terminal 16 are both arranged at the end corners, such that normal use of the first conductive region 11 and the second conductive region 12 is not affected.

The positive electrode connection terminal 15 and the negative electrode connection terminal 16 may be electrically connected to the positive and negative electrodes of the power source, respectively, via wires. When only one slipper is used for massage, two wires may be used for connection. Specifically, one end of one wire is electrically connected to the positive electrode connection terminal 15, and the other end of the wire is electrically connected to the positive electrode of the power source; and one end of the other wire is electrically connected to the negative electrode connection terminal 16, and the other end of the wire is electrically connected to the negative electrode of the power source. The two wires may be ordinary wires having only two connection terminals or Y-shaped wires having three connection terminals. When a pair of slippers are used for massage, two Y-shaped wires may be used for connection. Specifically, a connection terminal on one side, having only one connection terminal, of one wire is electrically connected to the positive electrode of the power source, and two connection terminals on the other side of the wire are respectively connected to the positive electrode connection terminals 15 of the two slippers; and a connection terminal on one side, having only one connection terminal, of the other wire is electrically connected to the negative electrode of the power source, and two connection terminals on the other side of the wire are respectively connected to the negative electrode connection terminals 16 of the two slippers. When the power source is turned on, the positive electrode of the power source transmits a current to the positive electrode connection terminals 15 via the Y-shaped wires. When the current flows through an intersection of the Y-shaped wires, the current is divided into two paths of currents respectively which flow to the positive electrode connection terminals 15 of the two slippers. The negative electrode connection terminals 16 of the two slippers transmit the two paths of currents to the negative electrode of the power source through the two connection terminals of the Y-shaped wires, and the two paths of currents converge into one current which flows to the negative electrode of the power source at the intersection point of the Y-shaped wires.

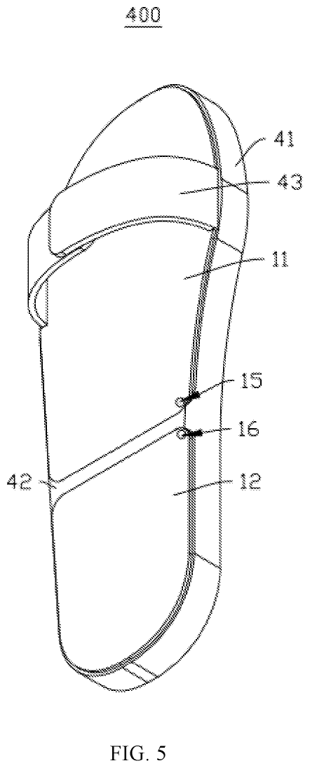

In some embodiments of the present disclosure, as illustrated in FIG. 5 which is a structural schematic view of another slipper according to an embodiment of the present disclosure, a protrusion 42 is formed on a side, for contacting with the sole, of the insulative shoe sole 41, and the protrusion 42 is positioned between the first conductive region 11 and the second conductive region 12. When the first conductive region 11 and the second conductive region 12 are separately and independently arranged on the surface of the insulative shoe sole 41, where the sole is in contact with the first conductive region 11 and the second conductive region 12, the position of the sole between the first conductive region 11 and the second conductive region 12 is in a suspended state, and the position is pressed greatly, such that the force on each position of the sole is uneven and discomfort is easily caused. A protrusion is formed on the insulative shoe sole 41 between the first conductive region 11 and the second conductive region 12, which supports the sole at this position, such that the force applied to each position of the sole is more uniform and the user feels more comfortable when using the foot electrical stimulation massager 100.

Some embodiments of the present disclosure further provide a massage apparatus. The massage apparatus includes the foot electrical stimulation massager according to any of the above embodiments.

Specifically, the massage apparatus may be a massage kit, such as a massage kit including a neck massager, a waist massager, a foot massager or the like, or may be a combined one-piece body massager.

It should be finally noted that the above-described embodiments are merely for illustration of the present disclosure, but are not intended to limit the present disclosure. Although the present disclosure is described in detail with reference to these embodiments, a person skilled in the art may also make various modifications to the technical solutions disclosed in the embodiments, or make equivalent replacements to a part of or all technical features contained therein. Such modifications or replacement, made without departing from the principles of the present disclosure, shall fall within the scope defined by the claims and the specification of the present disclosure. Especially, various technical features mentioned in various embodiments may be combined in any fashion as long as there is no structural conflict. The present disclosure is not limited to the specific embodiments described herein in this specification, but also includes all the technical solutions falling within the scope subject to the appended claims.

What is claimed is:

1. A foot electrical stimulation massager, comprising: a first conductive region and a second conductive region, the first conductive region and the second conductive region being configured to simultaneously apply electrical stimulation to a same sole;
wherein the first conductive region is connected to a positive electrode of a power source, the second conductive region is connected to a negative electrode of the power source, the first conductive region is configured to receive a current input by the positive electrode of the power source and transmit the current to the second conductive region via the sole upon transmitting the current to the sole thereon to apply the electrical stimulation, and the second conductive region is configured to output the current to the negative electrode of the power source.

2. The foot electrical stimulation massager according to claim 1, further comprising: a positive electrode connection terminal and a negative electrode connection terminal; wherein
the positive electrode connection terminal is electrically connected to the first conductive region, and the positive electrode connection terminal is configured to be connected to the positive electrode of the power source; and
the negative electrode connection terminal is electrically connected to the second conductive region, and the positive electrode connection terminal is configured to be connected to the negative electrode of the power source.

3. The foot electrical stimulation massager according to claim 2, further comprising: a base; wherein the first conductive region, the second conductive region, the positive electrode connection terminal, and the negative electrode connection terminal are all disposed on a surface of the base.

4. The foot electrical stimulation massager according to claim 1, wherein the first conductive region is in contact with a front plantar region of the sole in use, and the second conductive region is in contact with a heel region of the sole in use.

5. The foot electrical stimulation massager according to claim 2, further comprising: a third conductive region and a fourth conductive region, the third conductive region and the fourth conductive region being configured to simultaneously apply electrical stimulation to another sole;
wherein the third conductive region is electrically connected to the positive electrode connection terminal, and the fourth conductive region is electrically connected to the negative electrode connection terminal.

6. The foot electrical stimulation massager according to claim 2, wherein the positive electrode connection terminal and the negative electrode connection terminal are disposed between the first conductive region and the third conductive region.

7. The foot electrical stimulation massager according to claim 1, wherein the base is insulated, and the first conductive region and the second conductive region are respectively formed by arranging printed circuits on the base.

8. The foot electrical stimulation massager according to claim 7, wherein the printed circuits are made by printing silver paste, or the printed circuits are made by conductive rubber or graphene.

9. The foot electrical stimulation massager according to claim 1, wherein the first conductive region and the second conductive region are vertically arranged or horizontally arranged.

10. The foot electrical stimulation massager according to claim 1, wherein the first conductive region and the second conductive region together form a shape capable of accommodating a sole, to form a conductive region that simultaneously electrically stimulates the same sole.

11. The foot electrical stimulation massager according to claim 10, wherein a plurality of the conductive regions are arranged side by side on the foot electrical stimulation massager, forming a conductive region array for electrically stimulating to multiple pairs of soles.

12. The foot electrical stimulation massager according to claim 2, wherein the positive electrode connection terminal is arranged on the first conductive region, and directly electrically connected to the first conductive region; or the positive electrode connection terminal is arranged separately from the first conductive region, and electrically connected to the first conductive region via printed circuit traces or wire connections.

13. The foot electrical stimulation massager according to claim 2, wherein the negative electrode connection terminal is arranged on the second conductive region, and directly electrically connected to the second conductive region; or the negative electrode connection terminal is arranged separately from the second conductive region, and electrically connected to the second conductive region via printed circuit traces or wire connections.

14. The foot electrical stimulation massager according to claim 2, wherein the positive electrode connection terminal is a conductive snap-fit structure or a pin connection structure; the negative terminal is a conductive snap-fit structure or a pin connection structure.

15. The foot electrical stimulation massager according to claim 1, further comprising: a foot massage cushion; wherein the first conductive region and the second conductive region are disposed on an outer surface of the foot massage cushion.

16. The foot electrical stimulation massager according to claim 1, wherein the foot electrical stimulation massager is a slipper, the slipper comprising an insulative shoe sole; wherein the first conductive region and the second conductive region are both disposed on a surface of the insulative shoe sole, wherein the surface of the insulative shoe sole is close to the sole.

17. The foot electrical stimulation massager according to claim 16, wherein the positive electrode connection terminal is disposed at an end corner, facing towards the second conductive region, on the first conductive region; and
the negative electrode connection terminal is disposed at an end corner, opposite to the positive electrode connection terminal, on the second conductive region.

18. The foot electrical stimulation massager according to claim 16, wherein a protrusion is formed on a side of the insulative shoe sole, wherein the side of the insulative shoe sole is for contacting with the sole, and the protrusion is positioned between the first conductive region and the second conductive region.

19. A massage device, comprising: a foot electrical stimulation massager, wherein the foot electrical stimulation massager comprises:
a first conductive region and a second conductive region, the first conductive region and the second conductive region being configured to simultaneously apply electrical stimulation to a same sole;
wherein the first conductive region is connected to a positive electrode of a power source, the second conductive region is connected to a negative electrode of the power source, the first conductive region is configured to receive a current input by the positive electrode of the power source and transmit the current to the second conductive region via the sole upon transmitting the current to the sole thereon to apply the electrical stimulation, and the second conductive region is configured to output the current to the negative electrode of the power source.

20. The massage device according to claim 19, wherein the foot electrical stimulation massager further comprises:
a positive electrode connection terminal and a negative electrode connection terminal; wherein
the positive electrode connection terminal is electrically connected to the first conductive region, and the positive electrode connection terminal is configured to be connected to the positive electrode of the power source; and
the negative electrode connection terminal is electrically connected to the second conductive region, and the positive electrode connection terminal is configured to be connected to the negative electrode of the power source.
